(19) United States
(12) Patent Application Publication
(10) Pub. No.: US 2021/0196643 A1
(43) Pub. Date: Jul. 1, 2021

(54) PRODUCT

(71) Applicant: 4D Pharma Research Limited, Aberdeen (GB)

(72) Inventors: Christophe CARITE, Aberdeen (GB); Sophie DECLOMESNIL, Aberdeen (GB)

(21) Appl. No.: 17/120,856

(22) Filed: Dec. 14, 2020

Related U.S. Application Data
(63) Continuation of application No. PCT/GB2019/051720, filed on Jun. 19, 2019.

(30) Foreign Application Priority Data
Jun. 19, 2018 (GB) 1810061.0
Nov. 16, 2018 (GB) 1818740.1

(51) Int. Cl. A61K 9/48; A61K 35/745

(52) U.S. Cl. CPC A61K 9/4858; A61K 35/745

(57) ABSTRACT

The invention provides an enteric dosage form comprising a live biotherapeutic product.

### CROSS-REFERENCE

This application is a continuation of International Application No. PCT/GB2019/051720, filed Jun. 19, 2019, which claims the benefit of Great Britain Application No. 1810061.0, filed Jun. 19, 2018, and Great Britain Application No. 1818740.1, filed Nov. 16, 2018, all of which are hereby incorporated by reference in their entirety.

### TECHNICAL FIELD

The present invention relates to pharmaceutical products comprising a live biotherapeutic product. The invention also relates to processes for manufacturing such pharmaceutical products and kits comprising such products.

### BACKGROUND TO THE INVENTION

The human intestine is thought to be sterile in utero, but it is exposed to a large variety of maternal and environmental microbes immediately after birth. Thereafter, a dynamic period of microbial colonization and succession occurs, which is influenced by factors such as delivery mode, environment, diet and host genotype, all of which impact upon the composition of the gut microbiota, particularly during early life. Subsequently, the microbiota stabilizes and becomes adult-like. The human gut microbiota contains more than 500-1000 different phylotypes belonging essentially to two major bacterial divisions, the Bacteroidetes and the Firmicutes. The successful symbiotic relationships arising from bacterial colonization of the human gut have yielded a wide variety of metabolic, structural, protective and other beneficial functions. The enhanced metabolic activities of the colonized gut ensure that otherwise indigestible dietary components are degraded with release of by-products providing an important nutrient source for the host. Similarly, the immunological importance of the gut microbiota is well-recognized and is exemplified in germfree animals which have an impaired immune system that is functionally reconstituted following the introduction of commensal bacteria.

Dramatic changes in microbiota composition have been documented in gastrointestinal disorders such as inflammatory bowel disease (IBD). For example, the levels of Clostridium cluster XIVa bacteria are reduced in IBD patients whilst numbers of E. coli are increased, suggesting a shift in the balance of symbionts and pathobionts within the gut. Interestingly, this microbial dysbiosis is also associated with imbalances in T effector cell populations.

In recognition of the potential positive effect that certain bacterial strains can have on the mammalian gut, various strains have been proposed for use in the prevention, treatment and cure of various diseases. Examples of disclosures of the use of live bacterial organisms to treat physiological conditions include European Patent No. 1280541 (which discloses the use of hydrogenotrophic organisms in the treatment of a range of conditions including irritable bowel syndrome), European Patent No. 1448995 (which discloses the use of Bacteroides thetaiotamicron in the treatment of inflammatory diseases), European Patent Publication No. 2763685 (which discloses the use of Roseburia hominis as an immunoregulatory agent), European Patent No. 3209310 (which discloses the use of Enterococcus gallinarum as an anti-cancer therapy), and European Patent Publication No. 3206700 (which discloses the use of Bifidobacterium in the treatment of a range of autoimmune/inflammatory conditions, including severe asthma).

Such organisms are used as active principals in a class of pharmaceutical agents categorised by the US FDA as Live Biotherapeutic Products (“LBP”). In guidance published by the FDA in February 2012 and updated in 2016 (Guidance for Industry: Early Clinical Trials with Live Biotherapeutic Products: Chemistry, Manufacturing, and Control Information) LBP are defined as biological products that: 1) contain live organisms, such as bacteria; 2) are applicable to the prevention, treatment, or cure of a disease or condition of human beings; and 3) are not vaccines. It is further stated in the FDA's guidance document that (unlike probiotic products) LBP are subjected to the same rigorous scrutiny as pharmaceutical agents by regulatory bodies.

While LBP provide exciting and innovative therapies for a range of disease states, they remain challenging to formulate such that the active principles are viably delivered to the patient, especially after prolonged periods of storage prior to administration.

Those skilled in the art will be aware of active pharmaceutical ingredients (“APIs”) that have been in widespread use which require intestinal delivery, for example, to avoid a) being degraded in the acidic environment in the stomach e.g. proton pump inhibitors such as omeprazole, esomeprazole and pantoprazole or b) causing an irritant effect to the stomach, for example aspirin.

The skilled addressee will also be aware of formulation approaches to enable such APIs to be reliably delivered to the intestine even after prolonged periods of storage. One such approach is to formulate the API in enterically coated tablets, i.e. in tablet cores which are coated with one or more layers of gastroresistant material. When such tablets are orally administered to patients, the enteric coating material is not dissolved in the acidic medium of the stomach and thus release of the API contained in the stomach is prevented. However, once the tablets pass into the intestine and encounter the less acidic medium there, dissolution of the coating occurs and the API is released. Indeed, for APIs which exhibit optimal efficacy when delivered to specific regions of the intestine, pH-dependent coatings have been developed which dissolve and release API contained within when the coating is exposed to a medium having a specific pH. For example, the Eudragit® range of enteric polymers dissolve at pH 5.5 or above (for the L 30 D-55 and L 100-55 grades), at pH 6.0 or above (for the L 100 and L 12,5 grades) and above pH 7.0 (for the S 100, S 12,5 and FS 30 D grades).

These polymers are typically supplied as solutions or dispersions which can be used to coat tablet cores, or as powders which can be made up into solutions or dispersions by the tablet manufacturer and then used to coat the tablet cores.

An alternative, albeit related formulation approach for preparing APIs for intestinal delivery is to provide a formulation in a capsule, for example a soft or hard gelatin capsule, and then coat the capsule with enteric polymers such as those discussed above.

Additionally, to prevent the inadvertent egress of API from the coated capsule, it is common practice to additionally band capsules formed of two pieces to securely seal the capsule.

While such approaches have been successfully employed to formulate certain APIs for intestinal delivery on a commercial scale, it has been found that they are not necessarily applicable to more sensitive APIs, particularly LBP.

The present disclosure recognizes that one of the main reasons for this difficulty in formulating a viable product is the harsh conditions to which the live bacteria are subjected. In many LBP or probiotic formulations, the bacteria are provided in lyophilised form. This is achieved by freeze-drying, a process in which low temperatures and pressures are used to provide a dry, powdered product and the exposure of bacterial populations to such conditions results in a loss in viable organisms.

Even if a lyophilisation process can be optimised such that the loss of viable organisms in a bacterial population is minimised, the present disclosure recognizes that there are still other steps in the formulation process which can lead to further bacterial losses.

For example, typically after lyophilisation, the obtained powder is blended with excipients. Shear forces exerted by the mixing apparatus can inactivate bacteria. Additionally, for anaerobic organisms, the presence of oxygen in mixing apparatus can also cause viable cell loss.

For some applications, it is desirable to deliver the LBP to the intestine. As those skilled in the art will recognise, this can be achieved through the use of gastro-resistant coating/s, e.g. to tablet cores or to conventional capsules containing the LBP. It has been found that this step significantly adversely affects bacterial viability in some embodiments.

Without wishing to be bound by theory, it is contemplated in the present disclosure that this is due to ingress of solvent/s used during application of the enteric coating into the interior of the dosage form (e.g. the tablet core or the interior of the capsule) which kill the live bacteria and/or exposure of the products to elevated temperatures during drying of the coating.

The present disclosure also recognizes that, once formulated, risks to the viability of the bacterial population still exist. Prior to administration of the dosage form, it can be stored for months if not years prior to administration. Over time, subtle changes to the formulation can adversely affect the viable cell count, for example the take up of atmospheric moisture, or excipient incompatibility with the LBP, and these issues may only become apparent over prolonged periods.

Further, the present disclosure recognizes that the administration of the formulated product can also reduce viable cell count. Once ingested, the products encounter a highly acidic environment in the stomach before they reach the intestine. Formulating the products to protect the bacteria from the gastric environment in order to survive this stage in sufficient numbers can provide a significant therapeutic benefit as compared to a the corresponding bacteria found in nature. As explained above, one approach to enhance the gastro-resistance of APIs provided in enterically coated capsules is to band the capsules. However, in the context of LBP, banding processes (which require the use of solvents and/or elevated temperatures for drying) have now been linked to reductions in bacterial viability.

While a number of probiotic products have been commercialised which are enterically coated, the organisms in question are typically formulated at very high cell counts to allow for a reduction in viable organisms as a result of the effects outlined above. Those skilled in the art, however, will recognise that such an approach is not permissible for pharmaceutical products such as LBP, where the reliable delivery of a defined number of organisms is required by regulatory authorities.

Additionally, many probiotic formulations are based on bacteria with naturally high acid-resistance, such as Lactobacillus spp such as L. casei or those that can form protective spores, such as Bacillus spp such as B. clausii, which can then germinate in the relatively benign environment of the intestines. However, many LBP are not inherently acid resistant or do not sporulate and therefore require greater levels of protection.

One further difficulty of formulating LBP is that many therapeutically active organisms are anaerobic and the exposure of those organisms to air, either during the preparation of the products, or during storage (e.g. from ingress of air into the interior of products containing those organisms) is problematic and can result in a reduction of viable cell count.

Thus, although attempts have been made to produce pharmaceutical products comprising LBP for viable enteric delivery, the present disclosure recognizes challenges exist to the commercial scale production of such dosage forms. Accordingly, there remains a need for a formulation which can be used to deliver efficacious volumes of LBP to a patient with pharmaceutically acceptable reliability and which can be manufactured on a commercial scale, as well as processes for providing such formulations.

### SUMMARY OF THE INVENTION

According to a first aspect of the present invention, there is provided an orally administrable enteric dosage form comprising a live biotherapeutic product which i) comprises an antioxidant, wherein the antioxidant is cysteine, ii) does not comprise sodium carbonate or calcium carbonate, and/or iii) does not comprise maltodextrin.

The inventors have found that the above-mentioned selection (or in the case of options ii) or iii), the avoidance) of excipients advantageously and unexpectedly provides a dosage form which is shelf stable for at least 12 months, when stored at 2° C. to 8° C.

As used herein, the term shelf stable is used to mean that when the dosage form is stored at least 12 months in moisture-proof packaging at a temperature of 2° C. to 8° C., the viable cell count of the dosage form (CFU count per gram, excluding capsule shell (if present) or enteric coating (if present)) decreases by no more than 3 log. In certain embodiments, the viable cell count of the dosage form decreases by no more than 2 log when the dosage form is stored at least 12 months in moisture-proof packaging at a temperature of 2° C. to 8° C., and in certain other embodiments, the viable cell count of the dosage form decreases by no more than 1 log when the dosage form is stored at least 12 months in moisture-proof packaging at a temperature of 2° C. to 8° C. In embodiments of the invention, the viable cell count of the dosage form, when stored at 6 months in moisture-proof packaging at a temperature of 5° C. decreases by no more than 3 log. In certain embodiments, the viable cell count of the dosage form decreases by no more than 2 log over 6 months storage, in certain other embodiments, the viable cell count of the dosage form decreases by no more than 1 log over 6 months storage. In certain other embodiments, the viable cell count of the dosage form decreases by no more than 0.5 log over 6 months storage. In certain other embodiments, the viable cell count of the dosage form decreases by no more than 1 log over 21 months storage.

Regarding feature i) of the dosage form of the invention, numerous antioxidants are known to those of skill in the art of pharmaceutical formulation. The inventors have advantageously found that, as demonstrated in the examples which follow, cysteine outperforms conventionally used antioxidants such as ascorbic acid in maintaining a viable cell count of the therapeutically active bacteria.

In embodiments of the present invention, antioxidants in addition to cysteine can be employed. These include arginine, ascorbic acid (and salts and esters thereof e.g. ascorbyl palmitate, sodium ascorbate), butylated agents such as butylated hydroxyanisole or butylated hydroxytoluene, citric acid, erythorbic acid, fumaric acid, glutamic acid, glutathione, malic acid, methionine, monothioglycerol, pentetic acid, metabisulfite (such as sodium metabisulfite, potassium metabisulfite), propionic acid, propyl gallate, uric acid, sodium formaldehyde sulfoxylate, sulphite (e.g. sodium sulphite), sodium thiosulfate, sulphur dioxide, thymol, tocopherol (free or esterified), uric acid (and salts thereof) and salts and/or esters thereof.

While cysteine has been considered as an antioxidant in the formulation of certain pharmaceutical products, its use in the formulation of dosage forms comprising LBP is not known. The finding that cysteine can contribute to the long-term stabilisation of LBP is surprising given that cysteine has been known for many years to exhibit bacteriocidal effects. In this connection, reference may be made to Berglin et al. (Journal of Bacteriology, October 1982, 152(1), 81-8), especially the opening paragraph of that article which summarises the bacteriostatic effects of cysteine. Despite this effect, however, it has now been demonstrated in the examples which follow that cysteine does not exert its bacteriostatic effects on LBP, and (as mentioned above) actually enhances stability of the dosage forms of the invention.

Feature ii) of the present invention (the exclusion of sodium carbonate or calcium carbonate) is a further formulation approach which the inventors have unexpectedly identified as contributing to product stability permitting the delivery to patients of pharmaceutically acceptable levels of LBP.

A range of gas-evolving excipients have conventionally been used in the formulation of LBP. Indeed, the use of such excipients has been explicitly advocated in the scientific literature. For example, in a paper by Kim et al. (International Journal of Food Science and Technology 2017, 52, 519-530), the use of calcium carbonate as an encapsulant was advocated to enhance LBP survival under simulated gastric conditions and upon refrigerated storage. In Majkowska et al. (Polish Journal of Food and Nutrition Sciences, 2003, Vol. 12/53, SI 2, pp. 64-68), the use of calcium carbonate to supplement dietary calcium was suggested. In a product report (https://www.powerofprobiotics.com/Hyperbiotics-PRO-15.html) of a probiotic named PRO-15, commercialised by Hyperbiotics, it is explained that the use of sodium carbonate in that product is ‘to control the pH inside the tablet and around the disintegrating tablet in your intestine’. In an article reviewing commercially available probiotic formulations (https://livingwellnessblog.wordpress.com/2013/01/23/probiotic-paradox/), the author Dr David Peterson explained that ‘to increase survival probiotics not only should be refrigerated but acid proofed. Many companies do not use acid proofing. Others in an attempt to acid proof use calcium carbonate, an antacid. This neutralizes the sterilization effect of stomach acid and stimulation of the release of bile and pancreatic juices.’

In light of these recommendations, it is unsurprising that a plethora of commercialised LBP products comprise gas-evolving excipients such as sodium or calcium carbonate. Examples of such products include Hyperbiotics' PRO-15, Guts & Glory's Probiotic Power 60 Capsules, American Health's Probiotic CD and Kyodophilus' Kyolic to name a few.

Despite this, the inventors have unexpectedly found that the inclusion of excipients which evolve gas upon contact with acid actually has a deleterious effect on viability of the LBP when an enteric dosage form passes through the stomach, or a simulated gastric environment in in vitro testing. Without wishing to be bound by theory, it is believed that this is due to the ingress of trace amounts of acid when the dosage form is exposed to an actual or simulated gastric environment. In the event that this is the cause of destabilisation of the LBP, this is surprising for two reasons. Firstly, given that enteric dosage forms are prepared from materials which do not permit the ingress of biological medium until the dosage form reaches the intestine, the ingress of acidic medium from the gastric environment would not be expected. Secondly, even if the skilled person were to expect that minor amounts of acidic medium from the gastric environment can enter into the dosage form prior to the dosage form entering the intestine, he or she would be taught by the references above that the use of excipients such as sodium carbonate or calcium carbonate would actually protect against a loss of LBP viability caused by the acidic medium. However, it has now been found that the opposite is true.

Thus, in embodiments of the invention, the dosage form of the invention does not comprise sodium carbonate and/or calcium carbonate.

The third feature of the present invention (the exclusion of maltodextrin) is also beneficial to the provision of shelf stable dosage forms comprising LBP as the inventors have found that maltodextrin can be incompatible with LBP.

As explained above, the dosage form of the present invention is an orally administrable enteric dosage form, i.e. one which is capable of dissolution only in selective media (i.e. the intestinal environment) thus preventing release of its contents in the stomach. Such a gastroprotective dosage form as described herein can comprise an effective amount of an LBP (i.e. a live anaerobic bacteria) and an antioxidant, where the effective amount of the LBP in colony forming units (CFU) decreases by no more than 1 log in a simulated gastric environment. Gastroprotective properties can be determined by, for example: (a) exposing a dosage form described herein to an acid media at pH 1.2 for 30 minutes, (b) exposing the dosage form to an intestinal medium at pH 6.8 for 45 minutes, and (c) comparing the CFU after the exposing relative to prior to the exposing.

Those skilled in the art will be familiar with orally administrable enteric dosage forms and these include tablets, capsules, granules, and other micro- or nano-formulations, such as alginate encapsulated particles and in embodiments of the invention, the dosage form can be any of these. In some cases, enteric tablets or capsules are particularly preferred.

The skilled person will also be familiar with techniques for rendering dosage forms enteric. This is typically achieved by the application of enteric coatings to dosage forms, such as tablets or capsules, and enterically coated dosage forms constitute embodiments of the present invention. Enteric coating materials which can be employed in the present invention include polymers which dissolve at pH 5.5 or above (e.g. the Eudragit L 30 D-55 and L 100-55 grades), those which dissolve at pH 6.0 or above (for the Eudragit L 100 and L 12,5 grades) and/or those which dissolve at above pH 7.0 (for the Eudragit S 100, S 12,5 and FS 30 D grades). Additionally or alternatively, where the dosage form of the invention comprises a capsule, in addition to being enterically coated, the capsule can also be banded to prevent the ingress of gastric medium at the join between the two capsule halves.

In alternative embodiments, the dosage form can be an intrinsically enteric dosage form. The inventors have found that the use of such dosage forms, particularly intrinsically enteric capsules, provides a highly effective and straightforward approach to viably formulating LBP.

### DISCLOSURE OF THE INVENTION

As used herein, the term ‘intrinsically enteric capsule’ is used to refer to a capsule which is formed (either partially or totally) from material which dissolves when exposed to medium having a mildly acidic, neutral or basic pH, thus releasing the contents of the capsule into the medium. In embodiments of the invention, the intrinsically enteric capsule releases its contents when exposed to media having a pH of about 4.0 or above, about 4.5 or above, about 5.0 or above, about 5.5 or above, about 6.0 or above, about 6.5 or above or about 7.0 or above. Likewise, the term ‘enteric’ is used to refer to a material which dissolves upon exposure to media having a pH of about 4.0 or above, about 4.5 or above, about 5.0 or above, about 5.5 or above, about 6.0 or above, about 6.5 or above or about 7.0 or above.

Owing to its intrinsic enteric properties, the capsule does not require post-fill processing that could otherwise be potentially damaging to the LBP, for example, coating, drying and/or banding. Thus, in embodiments of the invention, the intrinsically enteric capsule does not comprise a continuous coating (i.e. one that covers the entirety of the capsule) and/or is unbanded.

The intrinsically enteric capsule can be single layered or multi-layered and/or be wholly or partly formed of gastrointestinal material which dissolves at the specific pH. In multilayered embodiments, one or more of the layers can be formed of enteric material which dissolves at the specific pH.

The intrinsically enteric capsule can be formed of any material/s which permit the total or partial dissolution of the capsule when exposed to medium having a mildly acidic, neutral or basic pH. In embodiments of the invention, the intrinsically enteric capsule can be formed partially or totally from fatty acids, waxes, shellac, plastics, plant fibers, enteric polymers or mixtures thereof. Enteric materials which can be employed in the present invention (either to produce intrinsically enteric dosage forms, or in alternative embodiments, enteric coatings) include, but are not limited to methacrylate polymers, methyl acrylate-methacrylic acid copolymers, methacrylic acid-methyl methacrylate copolymers, polyvinyl acetate phthalate, shellac, sodium alginate, zein, dextrins, amylose starch and starch derivatives, and cellulose and cellulose derivatives including hydroxy propyl methyl cellulose phthalate, hydroxy propyl methyl cellulose acetate succinate, cellulose acetate succinate, cellulose acetate trimellitate, cellulose acetate phthalate, or mixtures thereof. Plasticisers can also be comprised in the material from which the intrinsically enteric capsule is formed. Examples of materials that can be used in the production of intrinsically enteric capsules as well as methods for preparing such capsules are provided in European Patent No. 2722104, the contents of which are incorporated herein by reference. An example of an intrinsically enteric capsule is provided by Capsugel under the trade names enTRinsic DDT or ECDDT.

The capsules employed in the present invention can take any shape, form or construction provided that they can be closed to provide an enteric seal around the LBP comprised therein. For example, the capsules can be hard or soft. In embodiments of the invention, the capsule is a two part capsule or a multi part capsule (i.e. a capsule closed by coupling more than two parts).

For two part capsules or multi part capsules, the capsule parts can be closed by mechanically coupling the two or more parts of the capsule. Any form of mechanical interaction which results in the formation of a seal around the LBP can be employed. Examples of mechanical interaction that are envisaged include push-fit coupling, friction coupling and/or threaded coupling.

As used herein, the term “live biotherapeutic product” or “LBP” refers to a product that comprises live bacteria and is efficacious in the prevention, treatment or cure of a disease or condition, and is not a vaccine.

In embodiments of the invention, the LBP consists of or comprises anaerobic bacteria. In certain embodiments, the LBP comprises or consists of bacteria which are obligate anaerobes. As mentioned above, the formulation of anaerobic bacteria is particularly challenging using conventional enteric formulation approaches. However, the inventors have found that such organisms can be viably formulated according to the present invention.

Additionally or alternatively, the bacteria which can be formulated in accordance with the present invention can be hydrogenotrophic.

Organisms that can be formulated in accordance with the present invention include those belonging to the following genera: Enterococcus (e.g Enterococcus gallinarum, Enterococcus caselliflavus, Enterococcus faecalis, or Enterococcus faecium), Blautia (e.g. Blautia hydrogenotrophica, Blautia stercoris, Blautia wexlerae, Blautia coccoides or Blautia producta), Bacteroides (e.g. Bacteroides thetaiotaomicron, Bacteroides massiliensis, Bacteroides fragilis, Bacteroides ovatus, Bacteroides vulgatus, Bacteroides dorei, or Bacteroides copricola), Faecalibacterium (e.g. Faecalibacterium prausnitzii), Bariatricus (e.g. Bariatricus massiliensis), Bifidobacterium (e.g. Bifidobacterium breve, Bifidobacterium adolescentis or Bifidobacterium longum), Roseburia (e.g. Roseburia hominis, Roseburia intestinalis or Roseburia inulinivorans), Flavonifractor (e.g. Flavonifractor plautii), Anaerotruncus (e.g. Anaerotruncus colihominis), Parabacteroides (e.g. Parabacteroides distasonis, Parabacteroides goldsteinii, Parabacteroides merdae, or Parabacteroides johnsonii), Erysipelatoclostridium (e.g. Erysipelatoclostridium ramosum), Megasphaera (e.g. Megasphaera massiliensis, Megasphaera elsdenii), Pediococcus (e.g. Pediococcus acidilacticii), Eubacterium (e.g. Eubacterium contortum, fissicatena, Eubacterium eligens, Eubacterium hadrum, Eubacterium hallii, or Eubacterium rectale), Ruminococcus (e.g. Ruminococcus torques, Ruminococcus gnavus, or Ruminococcus bromii), Pseudoflavonifractor (e.g. Pseudoflavonifractor capillosus), Clostridium (e.g. Clostridium nexile, Clostridium hylemonae, Clostridium butyricum, Clostridium tertium, Clostridium disporicum, Clostridium bifermentans, Clostridium inocuum, Clostridium mayombei, Clostridium bolteae, Clostridium bartletti, Clostridium symbiosum or Clostridium orbiscindens), or Coprococcus (e.g. Coprococcus comes, or Coprococcus cattus), Bifidobacterium (e.g. Bifidobacterium breve, Bifidobacterium adolescentis or Bifidobacterium longum), Acetivibrio (e.g. Acetovibrio ethanolgignens), Dorea (e.g. Dorea longicatena) or Lachnospiraceae. Examples of such organisms include those disclosed in European Patent Nos. 1280541, 1448995, and 3209310, European Patent Publication No. 3206700, 2763685 and UK Patent Application No. 1423084.1, the contents of which are all incorporated herein by reference. Further examples of organisms that can be formulated according to the present invention include those disclosed in UK Patent Application Nos. 1510470.6, 1510468.0, 1510469.8, 1510466.4 and 1510467.2, the contents of which are all incorporated herein by reference. In embodiments of the invention, the dosage form does not comprise organisms belonging to Clostridium clusters IV or XIVa.

In embodiments of the invention, the LBP are not conventional probiotic bacteria, e.g. they do not belong to the genera Lactobacillus, Bifidobacterium and/or are not lactic acid bacteria.

In some embodiments, the LBP can comprise or consist of obligate anaerobic bacteria. In other embodiments, the LBP can comprise or consist of facultative anaerobic bacteria and/or microaerophilic bacteria.

In an exemplary embodiment, the LBP comprises or consists of non-sporulating bacteria.

In some embodiments, the dosage forms of the invention comprise one or more bacterial strains of a specific genus and do not contain bacteria from any other genera, or which comprise only de minimis or biologically irrelevant amounts of bacteria from another genera.

In some embodiments, the dosage forms of the invention comprise one or more bacterial strains of a specific species and do not contain bacteria from any other species, or which comprise only de minimis or biologically irrelevant amounts of bacteria from another species.

In certain embodiments, the dosage forms of the invention contain a single bacterial strain or species and do not contain any other bacterial strains or species. Such dosage forms can comprise only de minimis or biologically irrelevant amounts of other bacterial strains or species.

In some embodiments, the dosage forms of the invention comprise more than one bacterial strain. For example, in some embodiments, the dosage forms of the invention comprise more than one strain from within the same species (e.g. more than 1, 2, 3, 4, 5, 6, 7, 8, 9, 10, 15, 20, 25, 30, 35, 40 or 45 strains), and, optionally, do not contain bacteria from any other species.

In some embodiments, the dosage forms of the invention comprise less than 50 strains from within the same species (e.g. less than 45, 40, 35, 30, 25, 20, 15, 12, 10, 9, 8, 7, 6, 5, 4 or 3 strains), and, optionally, do not contain bacteria from any other species. In some embodiments, the dosage forms of the invention comprise 1-40, 1-30, 1-20, 1-19, 1-18, 1-15, 1-10, 1-9, 1-8, 1-7, 1-6, 1-5, 1-4, 1-3, 1-2, 2-50, 2-40, 2-30, 2-20, 2-15, 2-10, 2-5, 6-30, 6-15, 16-25, or 31-50 strains from within the same species and, optionally, do not contain bacteria from any other species. The invention comprises any combination of the foregoing.

In some embodiments, the dosage form comprises a microbial consortium. For example, in some embodiments, the dosage form comprises a specific bacterial strain as part of a microbial consortium. For example, in some embodiments, the dosage form comprises a bacterial strain which is present in combination with one or more (e.g. at least 2, 3, 4, 5, 10, 15 or 20) other bacterial strains from other genera with which it can live symbiotically in vivo in the intestine.

For example, in some embodiments, the dosage form comprises a specific bacterial strain in combination with a bacterial strain from a different genus. In some embodiments, the microbial consortium comprises two or more bacterial strains obtained from a faeces sample of a single organism, e.g. a human. In some embodiments, the microbial consortium is not found together in nature. For example, in some embodiments, the microbial consortium comprises bacterial strains obtained from faeces samples of at least two different organisms. In some embodiments, the two different organisms are from the same species, e.g. two different humans. In some embodiments, the two different organisms are an infant human and an adult human. In some embodiments, the two different organisms are a human and a non-human mammal.

In certain embodiments, the invention provides the above pharmaceutical dosage form, wherein the amount of the bacterial strain is from about 1×10³ to about 1×10¹¹ colony forming units per gram with respect to a weight of the dosage form (excluding the capsule body (if present) and any enteric coating (if present).

In some aspects, the pharmaceutical dosage form disclosed herein comprises one or more pharmaceutically acceptable excipients. Exemplary pharmaceutically acceptable excipients for the purposes of pharmaceutical compositions disclosed herein include, but are not limited to, binders, disintegrants, superdisintegrants, lubricants, diluents, fillers, flavors, glidants, sorbents, solubilizers, chelating agents, emulsifiers, thickening agents, dispersants, stabilizers, suspending agents, adsorbents, granulating agents, preservatives, buffers, coloring agents and sweeteners or combinations thereof. Examples of binders include microcrystalline cellulose, hydroxypropyl methylcellulose, carboxyvinyl polymer, polyvinylpyrrolidone, polyvinylpolypyrrolidone, carboxymethylcellulose calcium, carboxymethylcellulose sodium, ceratonia, chitosan, cottonseed oil, dextrates, dextrin, ethylcellulose, gelatin, glucose, glyceryl behenate, galactomannan polysaccharide, hydroxyethyl cellulose, hydroxyethylmethyl cellulose, hydroxypropyl cellulose, hypromellose, inulin, lactose, magnesium aluminum silicate, maltodextrin, methylcellulose, poloxamer, polycarbophil, polydextrose, polyethylene glycol, polyethylene oxide, polymethacrylates, sodium alginate, sorbitol, starch, sucrose, sunflower oil, vegetable oil, tocofersolan, zein, or combinations thereof. Examples of disintegrants include hydroxypropyl methylcellulose (HPMC), low substituted hydroxypropyl cellulose (L-HPC), croscarmellose sodium, sodium starch glycolate, lactose, magnesium aluminum silicate, methylcellulose, polacrilin potassium, sodium alginate, starch, or combinations thereof. Examples of a lubricant include stearic acid, sodium stearyl fumarate, glyceryl behenate, calcium stearate, glycerin monostearate, glyceryl palmitostearate, magnesium lauryl sulfate, mineral oil, palmitic acid, myristic acid, poloxamer, polyethylene glycol, sodium benzoate, sodium chloride, sodium lauryl sulfate, talc, zinc stearate, potassium benzoate, magnesium stearate or combinations thereof. Examples of diluents include talc, ammonium alginate, calcium carbonate, calcium lactate, calcium phosphate, calcium silicate, calcium sulfate, cellulose, cellulose acetate, corn starch, dextrates, dextrin, dextrose, erythritol, ethylcellulose, fructose, fumaric acid, glyceryl palmitostearate, isomalt, kaolin, lactitol, lactose, magnesium carbonate, magnesium oxide, maltodextrin, maltose, mannitol, microcrystalline cellulose, polydextrose, polymethacrylates, simethicone, sodium alginate, sodium chloride, sorbitol, starch, sucrose, sulfobutylether β-cyclodextrin, tragacanth, trehalose, xylitol, or combinations thereof.

Various useful fillers or diluents include, but are not limited to calcium phosphate, dibasic anhydrous, calcium phosphate, dibasic dihydrate, calcium phosphate tribasic, calcium sulphate, cellulose powdered, silicified microcrystalline cellulose, cellulose acetate, compressible sugar, confectioner's sugar, dextrates, dextrin, dextrose, fructose, kaolin, lactitol, lactose, lactose monohydrate, magnesium carbonate, magnesium oxide, maltodextrin, maltose, mannitol, microcrystailine cellulose, polydextrose, simethicone, sodium alginate, sodium chloride, sorbitol, starch, pregelatinized starch, sucrose, trehalose and xylitol, or mixtures thereof.

Examples of suitable lubricants include, but are not limited to, magnesium stearate, calcium stearate, zinc stearate, stearic acid, talc, glyceryl behenate, polyethylene glycol, polyethylene oxide polymers, sodium lauryl sulfate, magnesium lauryl sulfate, sodium oleate, sodium stearyl fumarate, DL-leucine, colloidal silica, and others as known in the art. In some embodiments a lubricant is magnesium stearate.

Various useful glidants include, but are not limited to, tribasic calcium phosphate, calcium silicate, cellulose, powdered, colloidal silicon dioxide, magnesium silicate, magnesium trisilicate, starch and talc, or mixtures thereof.

Pharmaceutically acceptable surfactants include, but are limited to both non-ionic and ionic surfactants suitable for use in pharmaceutical dosage forms. Ionic surfactants may include one or more of anionic, cationic or zwitterionic surfactants. Various useful surfactants include, but are not limited to, sodium lauryl sulfate, monooleate, monolaurate, monopalmitate, monostearate or another ester of olyoxyethylene sorbitane, sodium dioctylsulfosuccinate (DOSS), lecithin, stearyic alcohol, cetostearylic alcohol, cholesterol, polyoxyethylene ricin oil, polyoxyethylene fatty acid glycerides, and poloxamer.

In certain embodiments, the invention provides the above pharmaceutical composition, wherein said bacterial strain is lyophilised. In some cases, the bacterial strain is lyophilised in a process in which the bacterial strain is not exposed to temperatures greater than about 100° C., greater than about 70° C., greater than about 50° C. or greater than about 30° C.

In certain embodiments, the invention provides the above pharmaceutical composition, wherein when the composition is stored in a moisture tight container at 2° C. to 8° C. and the container is placed in an atmosphere having 50% relative humidity, the loss of the bacterial strain as measured in colony forming units (CFU) per gram is no greater than 3 log, no greater than 2 log or no greater than 1 log after a period of at least about 1 year, 1.5 years, 2 years, 2.5 years or 3 years. Additionally or alternatively, in embodiments of the invention, the composition, when stored in a moisture tight container at 5° C. and the container is placed in an atmosphere having 50% relative humidity, the loss of the bacterial strain as measured in colony forming units (CFU) per gram is no greater than 3 log, no greater than 2 log, no greater than 1 log or no greater than 0.5 log after a period of 6 months.

In certain embodiments, the dosage form contains the LBP in an amount of from about 1×10³ to about 1×10¹³ CFU/g, respect to the weight of the dosage form (excluding the capsule body (if present) and any enteric coating (if present), for example, from about 1×10⁴ to about 1×10¹² CFU/g, from about 1×10⁶ to about 1×10¹¹ CFU/g, from about 1×10⁸ to about 1×10¹², or from about 1×10⁸ to about 1×10¹⁰ CFU/g. In certain embodiments, the dosage form can comprise at least 1×10¹⁰ CFU/g, at least 1×10⁹ CFU/g, at least 1×10⁸ CFU/g, at least 1×10⁷ CFU/g, or at least 1×10⁶ CFU/g.

As mentioned above, the products of the present invention surprisingly maintain high levels of LBP viability following exposure to acidic media. In embodiments of the invention, the cell count of LBP contained within the products of the present invention, following exposure to a simulated gastrointestinal environment, namely a first medium having a pH of 1.2 for 30 minutes at 50 rpm paddle stirring followed by exposure to a second medium having a pH of 6.8 for 45 minutes at 120 rpm paddle stirring results in a reduction in viable cell count of 3 log or less, 2 log or less, or 1 log or less.

LBP viable cell count (e.g. to determine CFU/g) can be conducted using techniques known to those skilled in the art. For example, the CFU enumeration method can be carried out on lyophilised LBP. When a determination of CFU/g is made, the number of colony forming units per gram of composition present in the dosage form (i.e. excluding the capsule shell (if present) and enteric coating (if present) is determined.

The LBP present in the dosage form can be commensal, i.e. it is obtained from a donor (e.g. a human infant, child, adolescent or adult).

In embodiments of the invention, the dosage form comprises a biologically pure single strain of bacteria. As used herein the term “biologically pure” refers to a culture that comprises de minimis or biologically irrelevant levels of other strains of bacteria. In certain embodiments, the dosage form comprises less than about 1%, less than about 0.5%, less than about 0.2%, less than about 0.1%, less than about 0.05%, less than about 0.02% or less than about 0.01% as a proportion of the total number of bacterial cells of other bacterial species.

In alternative embodiments, the dosage form of the invention can comprise a plurality, e.g. 2, 3, 4, 5 or more than 5 strains of bacteria.

In addition to the LBP, the pharmaceutical products of the present invention can comprise one or more excipients including, for example diluents, stabilisers, growth stimulators, fillers, lubricants, glidants and the like.

In addition to the LBP (and in the case of feature i) of the dosage form as described above, cysteine), the dosage forms of the invention can comprise one or more pharmaceutically acceptable excipients. Examples of such suitable excipients can be found in the Handbook of Pharmaceutical Excipients. Acceptable excipients for therapeutic use are well known in the pharmaceutical art.

Examples of suitable carriers include lactose, starch, glucose, methyl cellulose, magnesium stearate, mannitol, sorbitol and the like.

Examples of suitable diluents include ethanol, glycerol and water. The choice of pharmaceutical carrier, excipient or diluent can be selected with regard to standard pharmaceutical practice.

The dosage forms can comprise any suitable binder(s), lubricant(s), suspending agent(s), coating agent(s), solubilising agent(s).

Examples of suitable binders include starch, gelatine, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavouring agents can be provided in the pharmaceutical composition.

Examples of preservatives include sodium benzoate, sorbic acid and esters of p-hydroxybenzoic acid.

Suspending agents can be also used.

In embodiments of the invention, the LBP is not microencapsulated.

In embodiments of the present invention, the product of the present invention can comprise a sugar for example a monosaccharide or disaccharide. Additionally or alternatively, the sugar can be a reducing sugar or non-reducing sugar. In such embodiments, where the products comprise a reducing sugar, non-reducing sugars can be excluded from the product, and vice versa. Examples of specific sugars that can be employed as excipients in the present invention include sucrose and trehalose.

The pharmaceutical products of the invention can further comprise a prebiotic. The term “prebiotic” means a non-digestible ingredient that beneficially affects the LBP by selectively stimulating the growth and/or activity of one or a limited number of bacteria. Examples of prebiotics include oligosaccharides, fructooligosaccharides and galactooligosaccharides.

The LBP (and optionally one or more of any excipients that are present) can be provided in the form of a lyophilisate. The lyophilizate can additionally comprise other excipients with which the LBP was lyophilised in order to protect the LBP during lyophilisation or to provide functional properties to the lyophilizate. As examples of excipients that can be present in the lyophilizate, these include mannitol, skim milk and bovine serum albumin (BSA), sucrose, trehalose and/or one of the other sugars identified above. A mixture of mannitol and sucrose as lyophilisation medium may be used.

In certain embodiments of the invention, the lyophilizate comprising the LBP can also comprise an antioxidant, (e.g. cysteine or a salt thereof). Additionally or alternatively, arginine, ascorbic acid (and salts and esters thereof e.g. ascorbyl palmitate, sodium ascorbate), butylated agents such as butylated hydroxyanisole or butylated hydroxytoluene, citric acid, erythorbic acid, fumaric acid, glutamic acid, glutathione, malic acid, methionine, monothioglycerol, pentetic acid, metabisulfite (such as sodium metabisulfite, potassium metabisulfite), propionic acid, propyl gallate, uric acid, sodium formaldehyde sulfoxylate, sulphite (e.g. sodium sulphite), sodium thiosulfate, sulphur dioxide, thymol, tocopherol (free or esterified), uric acid (and salts thereof) and salts and/or esters thereof can be present. In such embodiments, the remainder of the dosage form can be free of antioxidants.

An antioxidant employed in embodiments (for example cysteine), can be present as a salt. Examples of salts can include acetate, acrylate, adipate, alginate, aspartate, benzoate, benzenesulfonate, bisulfate, bisulfite, bitartrate, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate. metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate, undeconate, and xylenesulfonate.

The lyophilizate can be blended with additional excipients. Thus, in embodiments of the invention, the dosage form comprises i) a lyophilizate comprising the LBP and ii) one or more additional excipients. One or more of the additional excipients can also be provided in the lyophilizate.

In embodiments of the invention, the additional excipients can be any of the excipient disclosed herein, for example one or more of the antioxidants discussed herein, a carrier, diluent, binder, lubricant, suspending agent, coating agent, solubilising agent, stabiliser, growth stimulator, filler, lubricant and/or glidant.

It has been found that LBP viability is maintained upon storage where the moisture levels of the LBP are minimised. It is thought that this is because the presence of moisture increases LBP sensitivity to acidic degradation. Accordingly, in the products of the invention, the moisture content of the LBP in the dosage form of the invention is less than about 10000 ppm, less than about 5000 ppm, less than about 2000 ppm, less than about 1000 ppm, less than about 500 ppm, less than about 200 ppm or less than about 100 ppm.

According to a further aspect of the present invention, there is provided a process for preparing an orally administrable enteric dosage form comprising a live biotherapeutic product comprising i) providing a live biotherapeutic product and optionally one or more excipients, ii-a) filling the LBP and excipients into a capsule and closing the capsule to provide a filled capsule or ii-b) forming tablet cores comprising the LBP and excipients to provide tablet cores wherein the capsule is an intrinsically enteric capsule or wherein the process comprises the step of iii) applying an enteric coating to the filled capsule or tablet cores.

In embodiments of the invention, the one or more excipients comprises an antioxidant, wherein the antioxidant is cysteine, ii) does not comprise sodium carbonate or calcium carbonate, and/or iii) does not comprise maltodextrin.

In processes of the invention, the live biotherapeutic product can be provided by lyophilising bacteria alone or in combination with one or more excipients to produce a lyophilizate. The excipient/s provided in step i) of the process of the invention can be comprised in the lyophilizate, and/or be separate from the lyophilizate.

As mentioned above, the use of intrinsically enteric capsules in combination with LBP is advantageous as the use of conventional enteric coating process steps which can adversely affect LBP viability can be avoided. Thus, in processes of the present invention, the closed intrinsically enteric capsule is not subjected to coating, drying and/or banding steps.

Additionally, in the process of the present invention, the LBP may not be exposed to a temperature greater than about 50° C., greater than about 40° C. or greater than about 30° C. during steps i), ii-a) and/or ii-b).

As mentioned above, in embodiments of the invention, the LBP can comprise anaerobic bacteria. Accordingly, in the processes of the invention for preparing such products, steps i), ii-a) and/or ii-b) are operated in an inert (i.e. air-free) environment. The term “air-free environment” is used here to mean an environment comprising less than about 10000 ppm, less than about 5000 ppm, less than about 2000 ppm, less than about 1000 ppm, less than about 500 ppm, less than about 200 ppm or less than about 100 ppm of oxygen. Those skilled in the art will be aware of techniques for producing such a medium, for example flushing with an inert gas, e.g. nitrogen.

The dosage forms of the invention can be presented in a packaging material which can contain one or more dosage forms. The packaging material may, for example, comprise metal (e.g. aluminium) or plastic foil, such as a blister pack. Additionally, the products can be packaged in a bottle. Regardless of the specific type of packaging, the products of the present invention can be packaged in packaging material which is air and/or moisture impermeable containers. In some embodiments, a packaging can include packaging under reduced pressure.

In embodiments of the invention the pharmaceutical products are presented in the form of a kit comprising the products and instructions for use. The instructions for use can include instructions to store the products at temperatures less than about 20° C., less than about 15° C., or less than about 10° C., for example under refrigeration, e.g. at a temperature of about 2 to 8° C.

The present invention is further illustrated in the following examples.

### COMPARATIVE EXAMPLE 1

Lyophilizate comprising Blautia hydrogenotrophica and mannitol, sucrose and cysteine) was blended with calcium carbonate, cysteine and magnesium stearate was prepared in a temperature controlled clean room and filled into size 0 Enteric Capsule Drug Delivery Technology (ECDDT) capsules (Capsugel®).

The enteric coated capsule was then subjected to simulated gastric conditions (1 hour at an acidic media at pH 1.2) and a cell count conducted. The capsules ruptured, dispersing their contents into the acid medium. A count of viable LBP cells in that medium was conducted and it was found that a significant loss (over 3 log relative to the cell count carried out upon encapsulation) had occurred.

### EXAMPLE 2

### Storage Stability of Inventive Products

Mannitol, cysteine hydrochloride and magnesium stearate were weighed and mixed together in a temperature controlled clean room. The obtained blend was then mixed with lyophilizate comprising Blautia hydrogenotrophica, mannitol, sucrose and cysteine in a blender housed in a containment module in an inert atmosphere, at a temperature of <25° C. and at a relative humidity of <40%. The obtained blend was then filled into size 0 Enteric Capsule Drug Delivery Technology (ECDDT) capsules (Capsugel®) in the containment module with the obtained capsules then being packaged in moisture/air impermeable bags (PET/Alu/PA/PE).

The products were stored at 2 to 8° C. and cell counts were conducted upon i) encapsulation, and ii) thirteen months following encapsulation. Upon encapsulation, the viable cell count was 1.3×10¹¹ while at thirteen months, the count was 6.6×10¹⁰. The viable cell count per capsule was 4.7×10¹⁰ upon encapsulation and 2.0×10¹⁰ after 15 months of storage. No substantial loss in cell count (<1 log) was observed over periods in excess of 12 months demonstrating the storage stability of the products.

### EXAMPLE 3

### Dissolution of Inventive Products

Dosage forms prepared in accordance with Example 2 were exposed to acidic media (pH 1.2) for 2 hours under paddle stirring in accordance with the US Pharmacoepia <711>. No evidence of disintegration, rupture or content release of the capsules was observed. The products were then transferred to a simulated intestinal medium (pH 6.8) causing the capsules to rapidly disintegrate.

### EXAMPLE 4

### Gastroprotective Properties of Inventive Dosage Forms

Dosage forms prepared in accordance with Example 2 were exposed to acidic media (pH 1.2) for 30 minutes using the paddle method. They were then transferred to a simulated intestinal medium (pH 6.8) for 45 minutes. The example was ran under inert atmosphere (continuous sparging with nitrogen). Exposure of the products to the simulated intestinal medium resulted in the complete release of the LBP from the capsule and a cell count was performed. No substantial loss in cell count (<1 log) was observed. A cell count was also performed on the acidic medium and no release of LBP was detected, confirming the gastroprotective effect of the dosage forms of the present invention.

### EXAMPLE 5

### MRX518 with Ascorbic Acid vs Cysteine

Lyophilisate comprising Enterococcus gallinarum was prepared having the following compositions:

| | With Cysteine | With Ascorbic |
|---|---|---|
| | Antioxidant | Acid Antioxidant |
| | (% by weight of | (% by weight of |
| | lyophilizate | lyophilizate |
| Excipient | excipients) | excipients) |
| Sucrose | 84.0% | 84.0% |
| Mannitol | 14.5% | 14.5% |
| Cysteine | 1.5% | |
| Ascorbic Acid | | 1.5% |

The weight ratio of these lyophilisation excipients:Enterococcus gallinarum biomass in the lyophilizate was 1:1.

Counts of viable cells were carried out before the freeze drying process and afterwards, and the results are shown below. Both formulations were subjected to identical lyophilisation cycles:

| | With Cysteine | With Ascorbic |
|---|---|---|
| | Antioxidant | Acid Antioxidant |
| CFU/g of Frozen Biomass | 5.01 × 10¹¹ | 6.01 × 10¹¹ |
| (Prior to Lyophilisation) | | |
| CFU/g in Lyophilisation | 4.64 × 10¹¹ | 2.97 × 10¹¹ |
| (Post Lyophilisation) | | |
| Loss (log) | 0.03 | 0.31 |

The same assessment of viable cell count before and after lyophilisation was carried out at a different site and the results are shown below. Again, both formulations were subjected to identical lyophilisation cycles:

| | With Cysteine | With Ascorbic |
|---|---|---|
| | Antioxidant | Acid Antioxidant |
| CFU/g of Frozen Biomass | 7.34 × 10¹¹ | 7.29 × 10¹¹ |
| (Prior to Lyophilisation) | | |
| CFU/g in Lyophilisation | 7.19 × 10¹¹ | 2.76 × 10¹¹ |
| (Post Lyophilisation) | | |
| Loss (log) | 0.01 | 0.42 |

Thus, as is apparent, the use of cysteine as an antioxidant provided a significant and unexpected reduction in loss of viable cell count during the lyophilisation cycle.

### EXAMPLE 6

### Enterococcus gallinarum Long Term Stability

Cysteine-containing lyophilizate was then blended with an antioxidant-free excipient mixture and encapsulated within intrinsically enteric capsules. The resulting dosage forms were then packaged in alu/alu blister packaging and stored for a twelve month period at a temperature of 2 to 8° C.

A count of viable cells was conducted on the dosage form i) following encapsulation, ii) twelve months after encapsulation and iii) twenty one months after encapsulation.

Following encapsulation, a cell count of 1×10¹¹ CFU/g (7×10¹⁰ per capsule) was observed. After 12 months of storage conditions, a count of 4×10¹⁰ CFU/g (3×10¹⁰ per capsule) was recorded. The CFU per capsule recorded after 21 months of storage was 3×10¹⁰) This demonstrates that not only does the formulation protect LBP during lyophilisation, but it also permits a shelf-stable dosage form to be provided.

This shelf storage would not extend to the bacteria as naturally found in nature, but rather is a function of the inventive formulations as described herein

1.-30. (canceled)

31. A pharmaceutical composition comprising a viable cell count from about 1×10³ to about 1×10¹³ colony forming units per gram (CFU/g) of an anaerobic or microaerophilic bacteria strain with respect to a total weight of the pharmaceutical composition; and
an amount of an antioxidant sufficient for enhancing the shelf stability of the pharmaceutical composition, wherein the antioxidant comprises cysteine or ascorbic acid,
wherein the pharmaceutical composition is an orally administrable and enteric dosage form.

32. The pharmaceutical composition of claim 31, wherein the pharmaceutical composition does not comprise sodium carbonate, calcium carbonate, or maltodextrin.

33. The pharmaceutical composition of claim 31, wherein the viable cell count of the anaerobic or microaerophilic bacteria in colony forming units (CFU) decreases by no more than 1 log when the pharmaceutical composition is stored in moisture-proof packaging at a temperature of 2° C. to 8° C. for 12 months.

34. The pharmaceutical composition of claim 31, wherein the viable cell count of the anaerobic or microaerophilic bacteria in colony forming units (CFU) decreases by no more than 1 log when the pharmaceutical composition is stored in moisture-proof packaging at a temperature of from about 2° C. to about 8° C. for 21 months.

35. The pharmaceutical composition of claim 33, wherein the moisture-proof packaging comprises alu/alu blister packaging.

36. The pharmaceutical composition of claim 31, wherein the anaerobic or microaerophilic bacteria strain comprises Blautia hydrogenotrophica, Enterococcus gallinarum, Bacteroides thetaiotamicron, Roseburia hominis, or Bifidobacterium breve.

37. The pharmaceutical composition of claim 31, wherein the viable cell count comprises from about 1×10⁶ to about 1×10¹¹ CFU/g with respect to a weight of the pharmaceutical composition.

38. The pharmaceutical composition of claim 31, wherein the anaerobic or microaerophilic bacteria strain is lyophilized.

39. The pharmaceutical composition of claim 31, wherein the enteric dosage form comprises a tablet, a capsule, a granule, a micro-formulation, or a nano-formulation.

40. The pharmaceutical composition of claim 31, wherein the enteric dosage form is an intrinsically enteric dosage form.

41. The pharmaceutical composition of claim 40, wherein the intrinsically enteric dosage form is an intrinsically enteric capsule.

42. The pharmaceutical composition of claim 31, wherein the pharmaceutical composition comprises a single strain of the anaerobic or microaerophilic bacteria.

43. The pharmaceutical composition of claim 31, further comprising a pharmaceutically acceptable excipient.

44. The pharmaceutical composition of claim 43, wherein pharmaceutically acceptable excipient comprises a lyophilizate excipient comprising sucrose or mannitol.

45. The pharmaceutical composition of claim 31, wherein the antioxidant is cysteine.

46. The pharmaceutical composition of claim 45, wherein cysteine is present in the pharmaceutical composition in an amount of 0.75% by weight.

47. The pharmaceutical composition of claim 31, wherein the viable cell count is determined in a simulated gastric environment by:
(a) measuring the CFU of the viable cell count
(b) exposing the dosage form to an acid media at pH 1.2 for 30 minutes,
(c) exposing the dosage form to an intestinal medium at pH 6.8 for 45 minutes,
(d) measuring the CFU of the viable cell count, and
(e) comparing the CFU in the step (d) relative to the CFU in the step (a).

48. A method for preparing a pharmaceutical composition comprising an anaerobic or microaerophilic bacteria strain and one or more pharmaceutically acceptable excipients, wherein the pharmaceutical composition is an orally administrable and enteric dosage form, the method comprising:
(i) providing the anaerobic or microaerophilic bacteria strain and one or more pharmaceutically acceptable excipients, wherein the one or more pharmaceutically acceptable excipients comprises an antioxidant in an amount sufficient for enhancing shelf stability, wherein the antioxidant is cysteine or ascorbic acid, and
(ii-a) forming a tablet core comprising the anaerobic or microaerophilic bacteria strain and the one or more pharmaceutically acceptable excipients into a capsule and closing the capsule to provide the table core, or
(ii-b) filing the anaerobic or microaerophilic bacteria strain and the one or more pharmaceutically acceptable excipients into a capsule and closing the capsule to provide a filled capsule, wherein the capsule is an intrinsically enteric capsule, or
wherein the process comprises
(iii) applying an enteric coating to the filled capsule or the table core.

49. The method of claim 48, wherein the one or more pharmaceutically acceptable excipients does not comprise sodium carbonate, calcium carbonate, or maltodextrin.

50. The method of claim 48, wherein the anaerobic or microaerophilic bacteria strain comprises Blautia hydrogenotrophica, Enterococcus gallinarum, Bacteroides thetaiotamicron, Roseburia hominis, or Bifidobacterium breve.
